(19) United States
(12) Patent Application Publication
(10) Pub. No.: US 2026/0294229 A1
(43) Pub. Date: Oct. 1, 2026

(54) SURGICAL SYSTEMS INCLUDING VISUALIZATION ASSEMBLIES WITH INTEGRATED IRRIGATION AND INSTRUMENT GUIDES

(71) Applicant: Amplify Surgical, Inc., Foothill Ranch, CA (US)

(72) Inventors: Andy Wonyong Choi, Irvine, CA (US); Clark Hutton, Carlsbad, CA (US); Jin Hwa Eum, Abu Dhabi (AE)

(21) Appl. No.: 19/633,742

(22) Filed: Mar. 30, 2026

Related U.S. Application Data
(60) Provisional application No. 63/780,905, filed on Mar. 31, 2025.

(51) Int. Cl. A61B 1/313; A61B 1/00; A61B 1/015; A61B 1/018; A61B 17/34

(52) U.S. Cl. CPC A61B 1/3135; A61B 1/00128; A61B 1/00135; A61B 1/015; A61B 1/018; A61B 1/3132; A61B 17/3423

(57) ABSTRACT

Surgical systems including visualization assemblies with integrated irrigation and instrument guides are disclosed herein. In some embodiments, a surgical system includes an endoscopic visualization assembly at least partially insertable through a first port in the patient, and an irrigation cannula assembly at least partially insertable through a second port in the patient spaced apart from the first port. The visualization assembly can include a visualization instrument configured to endoscopically visualize a working space in the patient, a first visualization sheath configured to receive the visualization instrument, and a second visualization sheath configured to be fluidically coupled to an irrigation system and to receive the first visualization sheath. The second visualization sheath can include an instrument guide configured to receive and guide a first surgical instrument toward the working space. The irrigation cannula assembly can include a cannula configured to receive a second surgical instrument.

### CROSS-REFERENCE TO RELATED APPLICATION

The present application claims the benefit of U.S. Provisional Patent Application No. 63/780,905, filed Mar. 31, 2025, the disclosure of which is incorporated herein by reference in its entirety.

### TECHNICAL FIELD

The present technology relates generally to surgical systems and, in particular, to surgical systems including visualization assemblies with integrated irrigation and instrument guides.

### BACKGROUND

Individuals often suffer from damaged or displaced spinal discs and/or vertebral bodies due to trauma, disease, degenerative defects, or wear over an extended period of time. One result of this displacement or damage to a spinal disc or vertebral body may be chronic back pain. A common procedure for treating damage or disease of the spinal disc or vertebral body may involve partial or complete removal of an intervertebral disc. An intervertebral implant (commonly referred to as an interbody spacer or cage) can be inserted into the cavity created where the intervertebral disc was removed to help maintain height of the spine and/or restore stability to the spine. An interbody spacer may also provide a lordotic correction to the curvature of the spine. An example of an interbody spacer that has been commonly used is a fixed dimension cage, which typically is filled with bone and/or bone growth-inducing materials. Unfortunately, it may be difficult to implant the interbody spacer at the intended implantation site between vertebral bodies. Additionally, conventional surgical techniques can cause a significant amount of trauma at or near the implantation site (e.g., injury to nerve tissue), which can significantly increase recovery time and/or lead to patient discomfort.

Spinal nerve compression can be caused by narrowing of the spinal canal associated with arthritis (e.g., osteoarthritis) of the spine, degeneration of spinal discs, and thickening of ligaments. Arthritis of the spine often leads to the formation of bone spurs that can narrow the spinal canal and press on the spinal cord. In spinal disc degeneration, inner tissue of the disc can protrude through a weakened fibrous outer covering of the disc and can press on the spinal cord and/or spinal nerve roots. Ligaments located along the spine can thicken over time and press on the spinal cord and/or nerve roots. Unfortunately, spinal nerve compression can cause lower back pain, hip pain, and/or leg pain and may also result in numbness, depending on the location of the compressed nerve tissue. Surgical decompression procedures can be performed to enlarge the spinal canal, remove soft tissue, and remove other tissue contributing to nerve compression. Unfortunately, non-targeted tissue, such as nerve tissue, can be injured during such procedures. Accordingly, there is a need for improved surgical systems, visualization techniques, and related technologies for delivering a spinal implant and performing decompression procedures and/or other surgical procedures.

### BRIEF DESCRIPTION OF THE DRAWINGS

Features, aspects, and advantages of the presently disclosed technology may be better understood with regard to the following drawings.

FIG. 1 is a partially schematic view of a surgical system configured in accordance with embodiments of the present technology.

FIG. 2 is a top isometric view of a first visualization sheath configured in accordance with embodiments of the present technology.

FIG. 3 is a cross-sectional view of the first visualization sheath of FIG. 2.

FIG. 4 is an enlarged view of a proximal portion of the first visualization sheath of FIG. 2.

FIG. 5 is a top side view of a second visualization sheath configured in accordance with embodiments of the present technology.

FIG. 6 is a partially exploded view of the second visualization sheath of FIG. 5.

FIG. 7 is an isometric view of a sheath head configured in accordance with embodiments of the present technology.

FIG. 8 is a cross-sectional view of the sheath head of FIG. 7.

FIG. 9 is an isometric view of an irrigation fluid router of the second visualization sheath of FIG. 5 and configured in accordance with embodiments of the present technology.

FIG. 10 is a cross-sectional view of the irrigation fluid router of FIG. 9.

FIG. 11 is an isometric view of a sheath body configured in accordance with embodiments of the present technology.

FIG. 12 is a cross-sectional view of the sheath body of FIG. 11.

FIG. 13 is a cross-sectional view of a visualization assembly configured in accordance with embodiments of the present technology.

FIG. 14 is an enlarged cross-sectional view of the visualization assembly of FIG. 13.

FIG. 15 is an enlarged isometric view of the visualization assembly of FIG. 13.

FIG. 16 is a top isometric view of an obturator configured in accordance with embodiments of the present technology.

FIG. 17 is an enlarged view of a proximal end portion of the obturator of FIG. 16.

FIG. 18 is an enlarged view of a distal end portion of the obturator of FIG. 16.

FIG. 19 is a partially schematic view of another surgical system configured in accordance with embodiments of the present technology.

FIG. 20 is an isometric view of another second visualization sheath configured in accordance with embodiments of the present technology.

FIG. 21 is a partially exploded view of the second visualization sheath of FIG. 20.

FIGS. 22A-22D are top isometric, side, front, and top views, respectively, of an elongate member included in the second visualization sheath of FIG. 20 and configured in accordance with embodiments of the present technology.

FIG. 23 is an isometric view of an instrument assembly configured in accordance with embodiments of the present technology.

FIGS. 24A-24C are isometric, side, and top views, respectively, of a cannula included in the instrument assembly of FIG. 23 and configured in accordance with embodiments of the present technology.

FIG. 25 is a flowchart illustrating a method for performing a multi-portal spinal procedure on a patient in accordance with some embodiments of the present technology.

A person skilled in the relevant art will understand that the features shown in the drawings are for purposes of illustrations, and variations, including different and/or additional features and arrangements thereof, are possible.

### DETAILED DESCRIPTION

### I. Overview

Embodiments of the present technology are directed to surgical systems including visualization assemblies with integrated irrigation features and instrument guides. In a multi-portal surgical system, two or more incisions or ports can be formed in a patient. A first port can allow surgical instruments (e.g., implant delivery tools) to access a working space in the patient, and a second port can allow visualization instruments (e.g., endoscopes) to visualize the working space during operations. In some cases, it may be desirable to access the working space via multiple surgical tools simultaneously. To ensure that each of the multiple surgical tools can properly access the working space, it can be advantageous to insert the multiple surgical tools via different ports (e.g., to access the working space at different angles). Non-endoscopic procedures lead to several problems such as increasing the risk of infection and/or complications, prolonging the patient's recovery time, and/or the like. Single-port endoscopic procedures may not provide desired viewing of working spaces and positioning of instruments. Accordingly, there is a need for surgical systems that can allow multiple surgical instruments to access a working space simultaneously from different angles.

At least some embodiments of the present technology address at least some of the above-described issues. For example, embodiments of the present technology enable both a surgical instrument and a visualization instrument to access the working space via the same port. Therefore, multiple surgical instruments and a visualization instrument can simultaneously access the working space in a multi-portal surgical system: a first surgical instrument inserted through a first port, and a second surgical instrument and a visualization instrument both inserted through a second port. Surgical systems configured in accordance with embodiments of the present technology can also provide irrigation via either or both ports.

In some embodiments, a surgical system for performing multi-portal spinal procedures on a patient includes a visualization assembly at least partially insertable through a first port in the patient, and an instrument assembly at least partially insertable through a second port in the patient spaced apart from the first port. The visualization assembly can include a visualization instrument configured to visualize a working space in the patient, a first visualization sheath configured to receive the visualization instrument, and a second visualization sheath configured to receive the visualization instrument and the first visualization sheath. The second visualization sheath can include an instrument guide configured to guide a first surgical instrument to the working space through the first port and along the visualization assembly. The instrument assembly can include a cannula configured to guide a second surgical instrument to the working space through the second port.

In some embodiments, a surgical system includes an endoscopic visualization assembly configured to receive an endoscopic visualization instrument and deliver irrigation fluid to a working space within a patient. The endoscopic visualization assembly can include, for example, one or more sheaths, flow passageways, valves (e.g., one-way valves, duckbill valves), sealing members, locking features, or the like. In some embodiments, an irrigation flow passageway is located between inner and outer sheaths of the endoscopic visualization assembly. For example, the irrigation flow passageway can be formed by an inner surface of an outer sheath and the outer surface of an inner sheath. The outer sheath can include one or more flow diverters configured to direct irrigation fluid laterally outwardly away from the endoscopic visualization assembly. When the endoscopic visualization assembly is positioned within the patient, irrigation fluid can flow distally along the irrigation flow passageway and can exit via flow diverters to, for example, push tissue away from the endoscopic visualization assembly, enlarge working spaces, displace or push tissue away from viewing components of the endoscopic visualization instrument, etc. The flow diverters can include, for example, one or more apertures along a sidewall of the outer sheath.

The inner sheath can be a visualization sheath configured to receive a visualization instrument. The outer sheath can be a visualization sheath configured to couple to an irrigation system. The visualization sheath can include an inner sheath configured to fit in a passageway of the visualization sheath. In some embodiments, the visualization sheath can be configured to couple to a light source, such as a laser light source, and can include one or more optical elements (e.g., lenses, mirrors, fiber optics, etc.). The endoscopic visualization instrument can be an endoscope coupled to an imaging device configured to capture image data (e.g., still images, video, etc.).

In some embodiments, a method for performing a multi-portal spinal procedure on a patient includes (i) inserting a visualization assembly into a first port on the patient to access a working space in the patient. The visualization assembly can include a visualization instrument configured to visualize the working space in the patient, a first visualization sheath configured to receive the visualization instrument, and a second visualization sheath configured to receive the visualization instrument and the first visualization sheath. The method can further include (ii) inserting an instrument assembly into a second port on the patient spaced apart from the first port to access the working space, the instrument assembly including a cannula, (iii) inserting a first surgical instrument through the first port and along an instrument guide of the second visualization sheath, (iv) inserting a second surgical instrument through the second port and along the cannula of the instrument assembly, and (v) visualizing, via the visualization instrument of the visualization assembly, the working space. In some embodiments, the multi-portal procedure is a two-portal procedure in which a visualization instrument is positioned in a first port and one or more working instruments are positioned in a second port.

In some embodiments, a visualization assembly for use during a spinal procedure on a patient includes a first visualization sheath, a second visualization sheath, and a visualization instrument. The first visualization sheath can have a first channel. The second visualization sheath can have a second channel and an instrument guide. The second channel can be sized to receive at least a portion of the first visualization sheath therethrough. The instrument guide can be configured to guide a surgical instrument along a length of the second visualization sheath to a working space in the patient. The visualization instrument can be sized to at least partially extend through the first channel of the first visualization sheath and configured to visualize the working space.

Embodiments of the present technology are expected to provide a high degree of flexibility in performing multi-portal surgical (e.g., spinal) procedures. For example, unlike many existing surgical systems, surgical systems disclosed herein enable multiple surgical instruments to access (e.g., simultaneously) a working space in a patient before, while, and/or after visualizing the working space. The two surgical instruments can be inserted through different ports on the patient, allowing the working space to be accessed at different angles. This can be particularly beneficial if, for example, a surgeon is inserting a spinal implant through a first port and needs to push apart tissue through a second, different port. Moreover, irrigation can be provided through either or both the first and/or second ports. Accordingly, surgical systems configured in accordance with embodiments of the present technology can simultaneously or selectively (i) visualize the working space, (ii) provide irrigation of the working space through the first port and/or through the second port, and/or (iii) operate at the working space through the first port and/or through the second port.

In the figures, identical reference numbers identify generally similar, and/or identical, elements. Many of the details, dimensions, and other features shown in the figures are merely illustrative of particular embodiments of the disclosed technology. Accordingly, other embodiments can have other details, dimensions, and features without departing from the spirit or scope of the disclosure. In addition, those of ordinary skill in the art will appreciate that further embodiments of the various disclosed technologies can be practiced without several of the details described below.

### II. Select Embodiments of a Surgical System

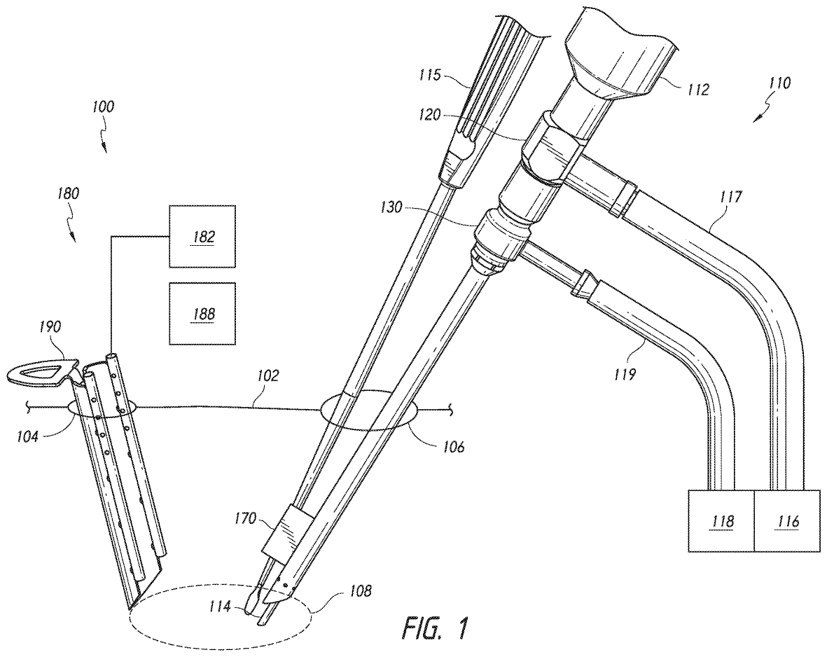

FIG. 1 is a partially schematic view of a surgical system 100 configured in accordance with embodiments of the present technology. The surgical system can include an instrument-guiding and/or endoscopic visualization assembly 110 and an instrument assembly 180 (also referred to as “the irrigation cannula assembly”). As shown and discussed in further detail herein, the visualization assembly 110 can be at least partially inserted through a first port 106 on a patient's skin 102 and the instrument assembly 180 can be at least partially inserted through a second port 104 on the patient's skin 102. Thus, the surgical system 100 can be a multi-portal surgical system in which the visualization assembly 110 and the instrument assembly 180 can simultaneously access a working space 108 in the patient from different angles.

The visualization assembly 110 can include a visualization instrument 112, a first visualization sheath 120, a second visualization sheath 130, a light source 116, and a first irrigation system or device 118. The visualization instrument 112 (e.g., an endoscope) can be coupled to each of the first visualization sheath 120 and the second visualization sheath 130. For example, the visualization instrument 112 can include an imaging device (e.g., camera, video device, etc.) seated at a proximal (e.g., top) end of the first visualization sheath 120, optical features (e.g., one or more lenses, reflectors, mirrors, etc.), fiber optics extending through each of the first visualization sheath 120, the second visualization sheath 130, and a distal tip 114 positionable at or near the working space 108.

As discussed in further detail herein, the first visualization sheath 120 and the second visualization sheath 130 can be removably coupled to one another to support the visualization instrument 112. As shown, each of the first visualization sheath 120 and the second visualization sheath 130 can have a generally tubular-shaped body that can be inserted through the first port 106. The first visualization sheath 120 can be operably coupled to the light source 116 via a first connector 117 (e.g., an optical connector, an optical cable). The second visualization sheath 130 can be operably (e.g., fluidically) coupled to the first irrigation device 118 via a second connector 119 (e.g., a fluid connector, a flexible tube, a hose, a fluid line, etc.). In operation, the light source 116 can provide light for the visualization instrument 112, and the first irrigation device 118 can provide irrigation fluid (e.g., saline, water) to irrigate the working space 108 before, during, and/or after use of the visualization instrument 112. In some embodiments, the light source 116 and the first irrigation device 118 form a singular, unitary visualization support unit. The unitary visualization support unit can include, for example, one or more features and/or components disclosed in U.S. Pat. Nos. 11,464,648 and 11,678,906, which are incorporated by reference in their entireties. U.S. Pat. Nos. 11,464,648 and 11,678,906 disclose displays, pumps, irrigation fluid supply systems, and surgical assistance systems that can be incorporated into the surgical system 100.

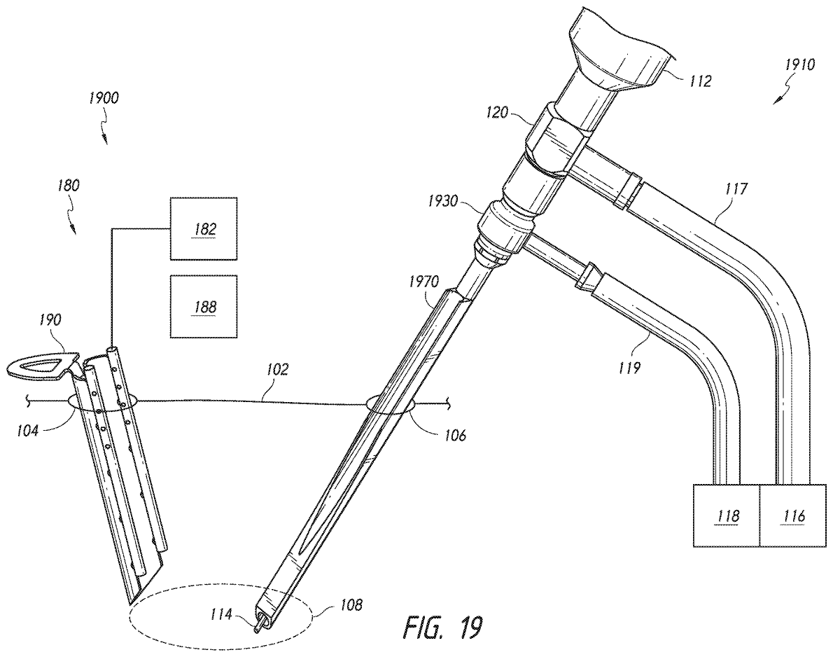

In some embodiments, the second visualization sheath 130 includes an instrument guide 170 (illustrated schematically as a box). As discussed in further detail herein, the instrument guide 170 can be used to securely hold, position, or otherwise guide a first surgical instrument 115 positioned outside of the second visualization sheath 130. As shown, the first surgical instrument 115 and the visualization assembly 110 can be simultaneously inserted through the first port 106. While the first surgical instrument 115 is illustrated as a spade knife in FIG. 1, it is appreciated that the first surgical instrument 115 can be another type of surgical instrument (e.g., a scalpel, a curette, a retractor, and/or the like). Specific embodiments of the instrument guide 170 are illustrated in and described with reference to FIGS. 19-22D.

The instrument assembly 180 can include a second irrigation device 182 and a cannula 190. The second irrigation device 182 can be coupled to the cannula 190 and can provide or suck irrigation fluid thereto or therefrom. While the second irrigation device 182 is illustrated as a separate and distinct component from the first irrigation device 118, in some embodiments, the first irrigation device 118 and the second irrigation device 182 can be the same device or otherwise form a unitary device. The cannula 190 can be inserted through the second port 104 and guide a second surgical instrument 188 toward the working space 108. In some embodiments, the second irrigation device 182 can output irrigation fluid that flows through the cannula 190 and into the working space 108 in the patient. The second irrigation device 182 can also provide suction to remove the irrigation fluid from the working space 108. For example, the second irrigation device 182 can alternatingly provide irrigation fluid and suction to sequentially deliver irrigation fluid into and suck irrigation fluid from the working space 108. In some embodiments, the second irrigation device 182 can deliver fluid into the working space 108 while the fluid is removed by the visualization assembly 110. During another portion of the procedure, the visualization assembly 110 can deliver irrigation fluid into the working space 108 and the cannula 190 can be used to remove the irrigation fluid.

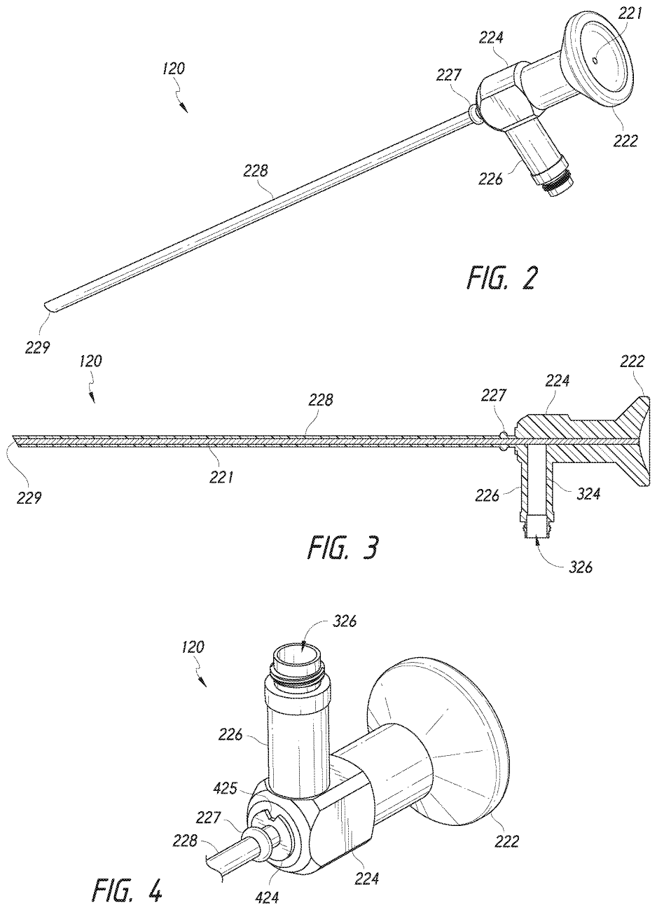

FIG. 2 is a top isometric view of the first visualization sheath 120. FIG. 3 is a cross-sectional view of the first visualization sheath 120. Referring to FIGS. 2 and 3 together, the first visualization sheath 120 can include an eyepiece 222, a sheath body 224, and an elongate member 228. The eyepiece 222 can define a proximal end portion of the first visualization sheath 120, and can support a portion of the visualization instrument 112 (e.g., a camera), as shown in FIG. 1. In the illustrated embodiment, the eyepiece 222 has a generally conical shape. In other embodiments, the eyepiece 222 can have other suitable shapes. The eyepiece 222 can also have an opening to a channel 221. As shown in FIG. 3, the channel 221 can extend through the entire length of the first visualization sheath 120. The channel 221 can be sized to receive a portion of the visualization instrument 112 (e.g., fiber optics) therethrough.

The sheath body 224 can be coupled to a distal end of the eyepiece 222. The sheath body 224 can include a laterally protruding tube 226 that defines a cavity 326 (FIG. 3) and an optical (e.g., transparent) block 324 (FIG. 3) extending between the cavity 326 and the portion of the channel 221 extending through the sheath body 224. The laterally protruding tube 226 can be releasably coupled to the first connector 117 (FIG. 1) via threads, press-fit, and/or other coupling mechanisms. In some embodiments, the cavity 326 can provide space for optical equipment included in the first connector 117 to be situated in. The transparent block 324 (e.g., a block made, in whole or in part, of glass, polycarbonate) can transmit light from the first connector 117 (generated by the light source 116 (FIG. 1)) to the channel 221, and the portion of the visualization instrument 112 extending through the channel 221 can carry the light toward the working space 108.

The elongate member 228 can extend distally from a distal end of the sheath body 224. In the illustrated embodiment, a distal tip 229 of the elongate member 228 is angled to, e.g., allow the visualization instrument 112 to visualize the working space 108 at an angle non-parallel to the elongate member 228. The angled distal tip 229 may also be shaped to facilitate insertion of the elongate member 228 into patient tissue (e.g., sharp enough to easily reach the working space 108, but blunt enough to avoid unintended tissue damage).

In some embodiments, the first visualization sheath 120 can also include a first coupling element 227. Also, as shown in FIG. 4, which is an enlarged bottom view of a proximal portion of the first visualization sheath 120, the first visualization sheath 120 can further include a second coupling element 424. In the illustrated embodiment, the first coupling element 227 is coupled to a proximal end portion of the elongate member 228 (e.g., adjacent to the sheath body 224) and has a substantially annular shape. The second coupling element 424 is coupled to a bottom surface of the sheath body 224 and is substantially disc-shaped with a cutout or notch 425. As discussed in further detail herein, the first coupling element 227 and the second coupling element 424 can each facilitate securing the first visualization sheath 120 to the second visualization sheath 130.

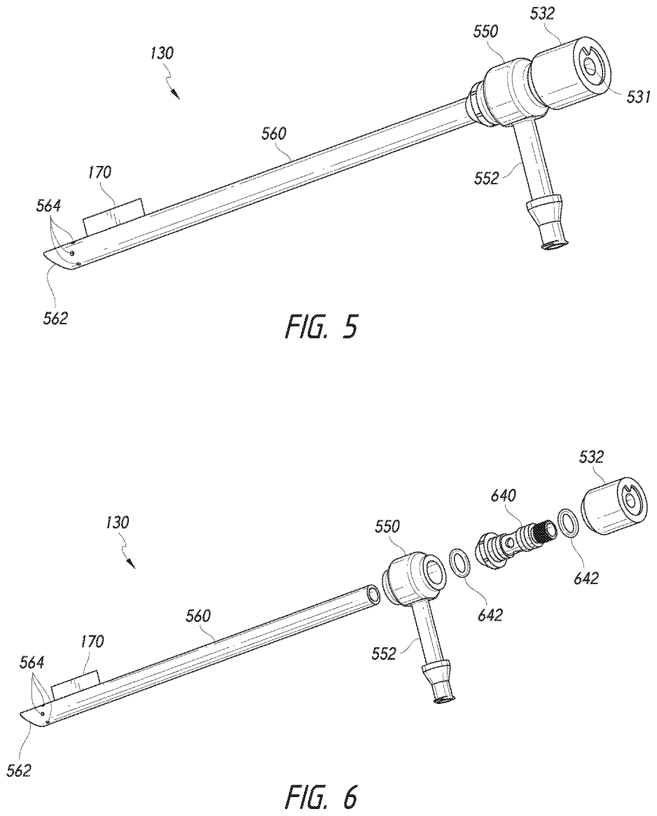

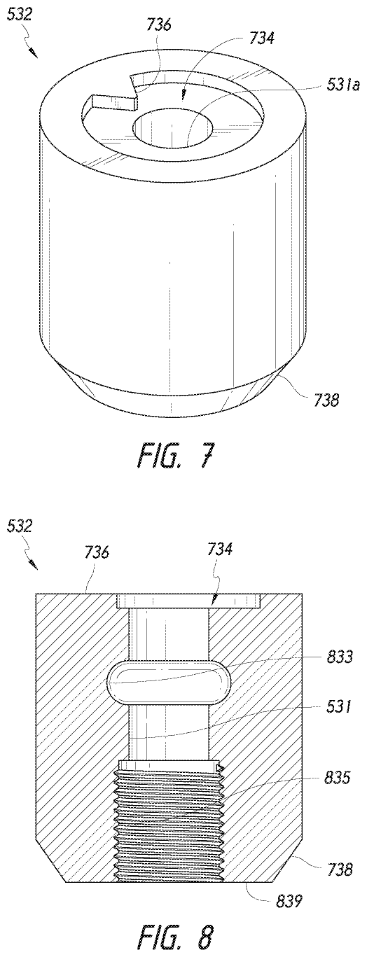

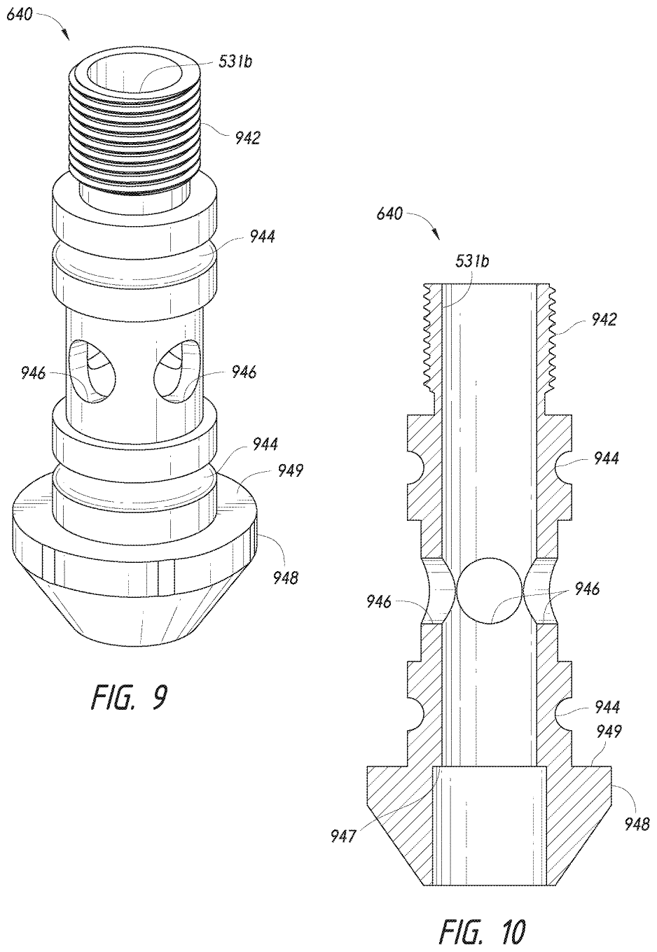

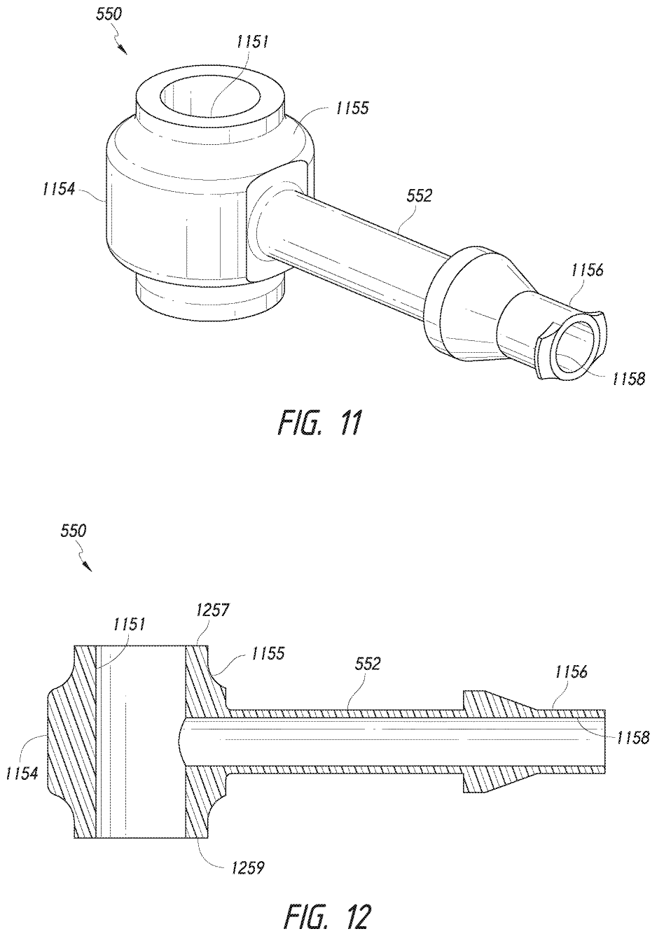

FIG. 5 is a top isometric view of the second visualization sheath 130. FIG. 6 is a partially exploded view of the second visualization sheath 130. Referring to FIGS. 5 and 6 together, the second visualization sheath 130 can include a sheath head 532, an irrigation fluid router 640 (FIG. 6), one or more sealing members 642 (FIG. 6), a sheath body 550, an elongate member 560, and the instrument guide 170. The sealing members 642 can include compressible members, O-rings, and/or components configured to form (e.g., automatically), for example, fluid-tight and/or liquid-tight seals, hermetic seals, etc. The sheath head 532 (also referred to herein as “the coupler head 532”) can define a proximal end portion of the second visualization sheath 130, and can interface with the sheath body 224 of the first visualization sheath 120, as shown in FIG. 1. The sheath head 532 can also define an opening to a passageway or channel 531 (“channel 531”) that extends along the length of the second visualization sheath 130. The channel 531 can be shaped and sized to receive the elongate member 228 of the first visualization sheath 120. The sheath head 532 is illustrated in and described in greater detail with reference to FIGS. 7 and 8. The irrigation fluid router 640 can be a flow director, a manifold, and/or the like, and can be coupled to the sheath head 532 and disposed at least partially within the sheath body 550 (also referred to herein as “the connector body 550”). The sealing members 642 can be disposed between the irrigation fluid router 640 and the sheath body 550. The irrigation fluid router 640 is illustrated in and described in greater detail with reference to FIGS. 9 and 10. The sheath body 550 can be coupled to a distal end of the sheath head 532, as shown in FIG. 5. The sheath body 550 can include a laterally protruding tube 552. The sheath body 550 is illustrated in and described in greater detail with reference to FIGS. 11 and 12.

The elongate member 560 can extend from the sheath body 550 distally and can have a distal tip 562. Like the distal tip 229 of the elongate member 228 (FIGS. 2 and 3), the distal tip 562 of the elongate member 560 can be angled. The elongate member 560 can also include one or more apertures 564 positioned at or near the distal tip 562. The apertures 564 can be circular in shape and distributed circumferentially around the elongate member 560. As discussed in further detail herein, the apertures 564 can serve as exits for irrigation fluid. The instrument guide 170 (illustrated schematically) can be coupled to the elongate member 560 near the distal tip 562, along the length of the elongate member 560, and/or otherwise coupled to or formed on the elongate member 560.

FIG. 7 is an isometric view of the sheath head 532. As shown, the sheath head 532 can have a substantially cylindrical form factor with a tapered lower sidewall portion 738. The top or proximal end of the sheath head 532 can define a recess 734 and have a protrusion 736 extending at least partially into the recess. In some embodiments, the shape and size of the recess 734, accounting for the protrusion 736, matches or otherwise corresponds to the shape and size of the second coupling element 424, accounting for the notch 425, of the first visualization sheath 120. Thus, when the first visualization sheath 120 and the second visualization sheath 130 are assembled together, the bottom of the sheath body 224 (FIG. 4) can contact or otherwise interface with the top of the sheath head 532, and the second coupling element 424 can fit in the recess 734 such that the protrusion 736 is received in the notch 425. Also, the sheath head 532 can include a channel portion 531a that, when the second visualization sheath 130 is assembled, forms part of the channel 531.

FIG. 8 is a cross-sectional view of the sheath head 532. As shown, the channel portion 531a can include an internal annular groove 833 and threads 835. The internal annular groove 833 can be shaped and sized to receive the first coupling element 227 (FIGS. 2-4) of the first visualization sheath 120. Thus, when the first visualization sheath 120 and the second visualization sheath 130 are assembled together, the first coupling element 227 can be secured in the internal annular groove 833. In some embodiments, the first coupling element 227 is composed of a malleable and/or flexible material (e.g., silicone, rubber) such that the user can push the first coupling element 227 into the channel portion 531a and ultimately into the internal annular groove 833. As discussed in further detail herein, the threads 835 can be used to couple the sheath head 532 to the irrigation fluid router 640. Moreover, the sheath head 532 can have a generally flat bottom surface 839 that, when the second visualization sheath 130 is assembled, abuts against the irrigation fluid router 640 and the sheath body 550.

FIGS. 9 and 10 are isometric and cross-sectional views, respectively, of the irrigation fluid router 640. Referring to FIGS. 9 and 10 together, the irrigation fluid router 640 can include threads 942, one or more annular grooves 944, one or more irrigation fluid apertures 946, and a tapered lower portion 948. The threads 942 can be formed at a top or proximal end portion of the irrigation fluid router 640, and can correspond to the threads 835 (FIG. 8). Therefore, the irrigation fluid router 640 can be coupled to the sheath head 532 by at least partially inserting the irrigation fluid router 640 into the channel portion 531a of the sheath head 532 and engaging the threads 942 with the threads 835. The one or more annular grooves 944 can be shaped and sized to receive corresponding ones of the one or more sealing members 642 (FIG. 6). In the illustrated embodiment, the irrigation fluid router 640 includes two annular grooves 944, one above the irrigation fluid apertures 946 and another below the irrigation fluid apertures 946.

In the illustrated embodiment, the irrigation fluid router 640 includes four circular irrigation fluid apertures 946 positioned between the two annular grooves 944 and equally spaced apart from one another circumferentially around the irrigation fluid router 640. Each of the irrigation fluid apertures 946 can be in fluid communication with a channel portion 531b that extends through the length of the irrigation fluid router 640 and that, when the second visualization sheath 130 is assembled, forms part of the channel 531. In some embodiments, the irrigation fluid apertures 946 can include one or more nozzles, baffles, or flow guides configured to direct irrigation fluid in a desired direction. The number, configuration, and placement of the irrigation fluid apertures 946 can be selected based on the procedure to be performed.

In some procedures, multiple second visualization sheaths 130 can be used. For example, a second visualization sheath 130 with relatively large irrigation fluid apertures 946 can be used during surgical steps with high irrigation flow rates. Another second visualization sheath 130 with relatively small irrigation fluid apertures 946 can be used during surgical steps with low irrigation flow rates. The tapered lower portion 948 can have a top annular surface 949 that, when the second visualization sheath 130 is assembled, abuts the sheath body 550. Moreover, referring specifically to FIG. 10, the tapered lower portion 948 can include an inner annular lip 947 in the channel portion 531b. When the second visualization sheath 130 is assembled, the elongate member 560 can abut the lip 947.

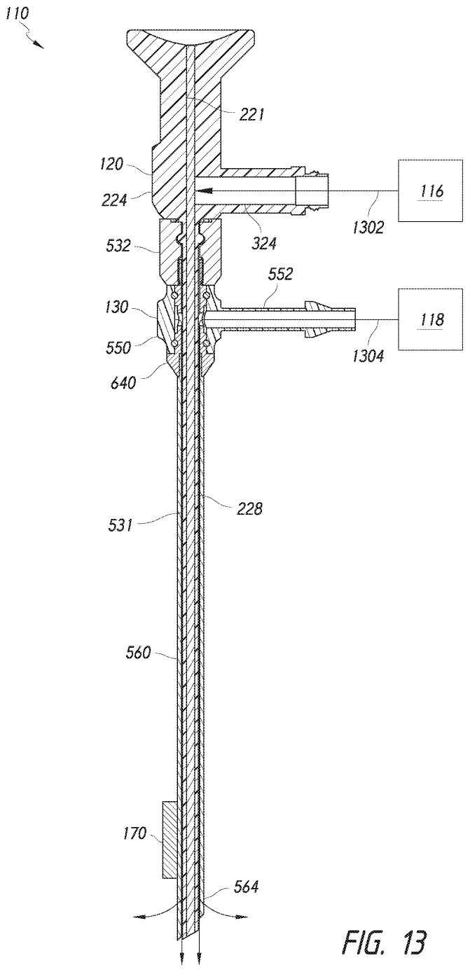

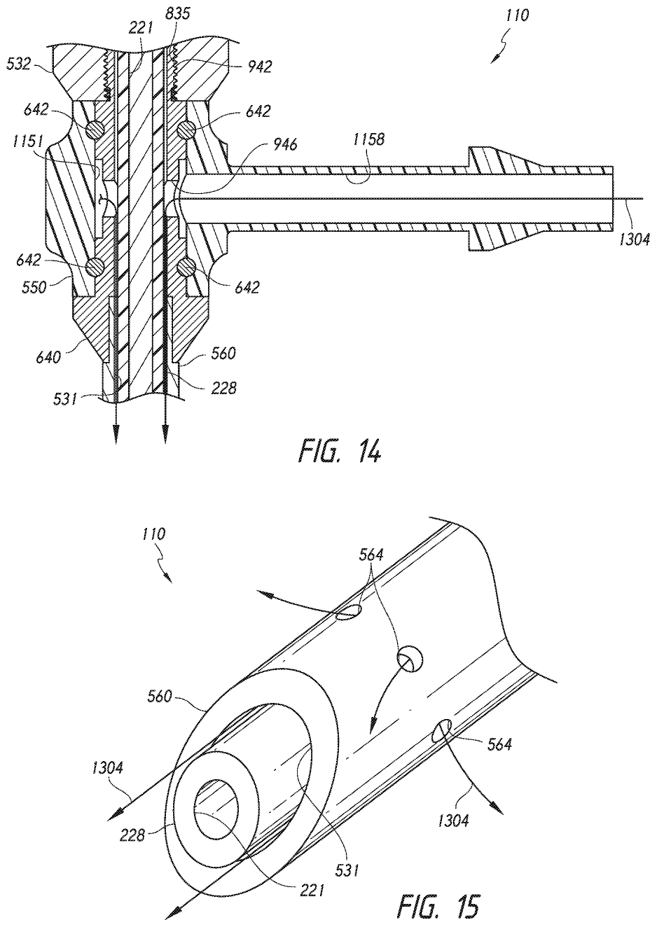

FIGS. 11 and 12 are isometric and cross-sectional views, respectively, of the sheath body 550. Referring to FIGS. 11 and 12 together, the sheath body 550 can include a housing 1154, the laterally protruding tube 552, and an irrigation fluid receiver 1156. The housing 1154 can define an aperture 1151 extending vertically therethrough. As discussed in further detail below with reference to FIGS. 13 and 14, the aperture 1151 can be shaped and sized to receive at least a portion of the irrigation fluid router 640. When the second visualization sheath 130 is assembled, a top surface 1257 of the housing 1154 can abut the bottom surface 839 (FIG. 8) of the sheath head 532, and a sloped surface 1155 of the housing 1154 can form a neck portion with the tapered lower sidewall portion 738 (FIGS. 7 and 8). Also, a bottom surface 1259 of the housing 1154 can be seated on the top annular surface 949 of the irrigation fluid router 640. The laterally protruding tube 552 can extend from the housing 1154 in a direction substantially perpendicular to the aperture 1151. The irrigation fluid receiver 1156 can be disposed at a distal end of the laterally protruding tube 552, and can be shaped to couple to, e.g., the second connector 119 (FIG. 1). In particular, the laterally protruding tube 552 and the irrigation fluid receiver 1156 can define a channel 1158 that is open at the irrigation fluid receiver 1156 and in fluid communication with the aperture 1151.

FIG. 13 is a cross-sectional view of the visualization assembly 110 in an assembled state. As shown, the sheath body 224 of the first visualization sheath 120 can rest on the sheath head 532 such that the second coupling element 424 (FIG. 4) is disposed in the recess 734 (FIG. 7), and the protrusion 736 (FIG. 7) is situated in the notch 425 (FIG. 4). Also, the elongate member 228 of the first visualization sheath 120 can extend through the channel 531 of the second visualization sheath 130. The sheaths 120, 130 can be coupled together by at least one of a mechanical clamp, a magnetic coupler, or a pin. In particular, the first coupling element 227 (FIGS. 2-4) can be disposed in the internal annular groove 833 (FIG. 8). Thus, the first coupling element 227 and the second coupling element 424 can fix both the relative position and orientation of the first visualization sheath 120 and the second visualization sheath 130. The elongate member 228 may extend past the apertures 564 of the elongate member 560, as shown, or may not. Moreover, the irrigation fluid router 640 can be positioned at least partially within or inside the aperture 1151 (FIGS. 11 and 12) of the sheath body 550.

The light source 116 can be operably coupled to the first visualization sheath 120 and can provide light 1302 (illustrated as an arrow) through the transparent block 324. Thus, the light 1302 can reach the channel 221 of the first visualization sheath 120, and the visualization instrument 112 (FIG. 1) can carry the light 1302 toward and past the distal ends of both the first visualization sheath 120 and the second visualization sheath 130. The first irrigation device 118 can be operably coupled to the second visualization sheath 130 and can provide irrigation fluid 1304 (illustrated as arrows) thereto. Delivery of the irrigation fluid 1304 is described in greater detail below with reference to FIG. 14. While illustrated as separate components, in some embodiments, the light source 116 and the first irrigation device 118 form a singular, unitary visualization support unit.

FIG. 14 is an enlarged cross-sectional view of the visualization assembly 110. As shown, the irrigation fluid router 640 can be coupled to the sheath head 532 (e.g., via the threads 835 (FIG. 8) and the threads 942 (FIGS. 9 and 10)), and a portion of the irrigation fluid router 640 can be disposed in the aperture 1151 of the sheath body 550. In particular, the bottom surface 1259 (FIG. 12) of the sheath body 550 can be seated on the top annular surface 949 (FIG. 9) of the irrigation fluid router 640. Also, as shown, the sealing members 642 can be positioned in corresponding ones of the annular grooves 944 (FIGS. 9 and 10), and can be compressed between the irrigation fluid router 640 and the sheath body 550 surrounding the irrigation fluid router 640. The various components of the visualization assembly 110 can be coupled together via threads, press-fit, adhesives, welding, fasteners, and/or other suitable coupling mechanisms, and/or can be integrally formed.

When the irrigation fluid 1304 is delivered via the channel 1158 of the sheath body 550, the irrigation fluid 1304 can flow into the gap between the irrigation fluid router 640 and the aperture 1151 of the sheath body 550. The compressed sealing members 642 can prevent the irrigation fluid 1304 from flowing out of the second visualization sheath 130 (e.g., via the gap between the sheath body 550 and the irrigation fluid router 640) and can ensure that the irrigation fluid 1304 remains therein. Subsequently, the irrigation fluid 1304 can flow into the irrigation fluid apertures 946 of the irrigation fluid router 640 and into the annular gap between the elongate member 228 of the first visualization sheath 120 and the channel 531 of the second visualization sheath 130. Thus, the irrigation fluid 1304 can flow down the annular gap between the elongate member 228 and the elongate member 560. Notably, the irrigation fluid 1304 does not flow into the channel 221 of the first visualization sheath 120.

FIG. 15 is an enlarged isometric view of the visualization assembly 110 and, in particular, the distal ends of the elongate member 228 and the elongate member 560. As indicated by arrows, the irrigation fluid 1304 can flow through the annular gap between the elongate member 228 and the elongate member 560 (e.g., through the channel 531), and exit via (i) the apertures 564 of the elongate member 560 and/or (ii) the distal end of the channel 531. In some embodiments, the apertures 564 can be evenly or unevenly spaced about the circumference of the elongate member 560. The distance between the distal tip 562 and the apertures 564 can be selected based on the location of tissue to be pushed away from the visualization assembly 110. When irrigation flows distally, the irrigation fluid 1304 can flow through the apertures 564 away from the second visualization sheath 130 in, for example, a radial direction. The irrigation fluid 1304 can flow toward surrounding tissue to urge tissue away from a distal tip of the endoscope, thereby forming an enlarged visualization space within the patient. The flow rate of the irrigation fluid 1304 can be increased or decreased to increase or decrease the fluid pressure in the working space. In some embodiments, the apertures 564 can include one or more nozzles, baffles, or flow guides configured to direct the irrigation fluid 1304 in a desired direction. The number, size, configuration, and placement of the apertures 564 can be selected based on the procedure to be performed.

The combination of the apertures 564 and the distal end of the channel 531 can provide a wider irrigation field than either alone. Accordingly, the visualization assembly 110 can provide irrigation of the working space 108 (FIG. 1) before, while, and/or after the visualization instrument 112 (extending out through the channel 221; not shown in FIG. 15) visualizes the working space 108.

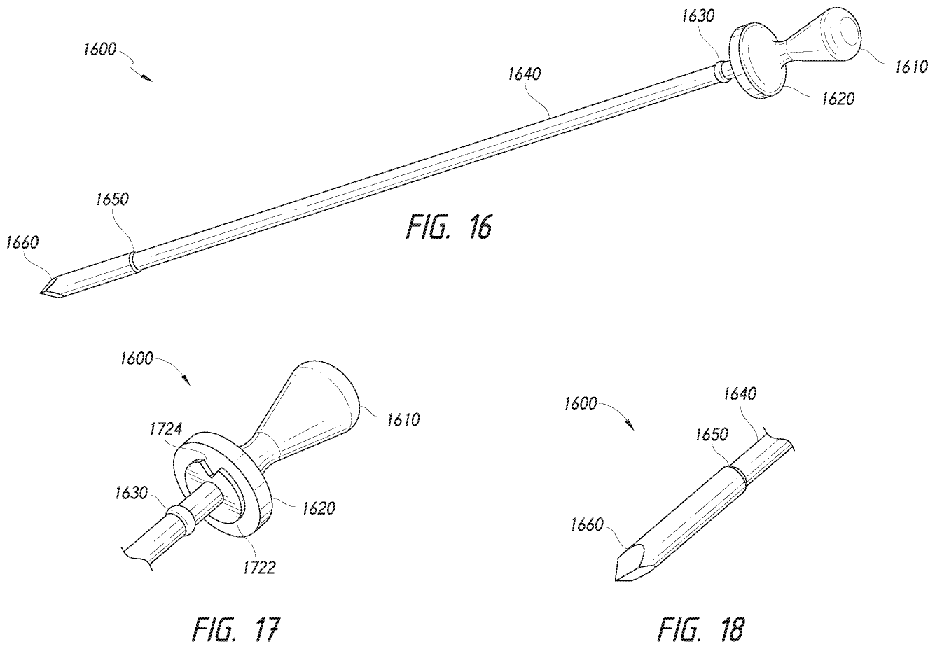

FIG. 16 is a top isometric view of an obturator 1600 configured in accordance with embodiments of the present technology. As discussed in further detail herein, the obturator 1600 can be part of the surgical system 100 (FIG. 1) and can be inserted in the channel 531 of the second visualization sheath 130 instead of the first visualization sheath 120. The obturator 1600 can include a handle 1610, a flange 1620, a first coupling element 1630, an elongate member 1640, and a piercing portion 1660. The handle 1610 can be shaped to provide an ergonomic grip of the obturator 1600. The flange 1620 can be distal to the handle 1610 and can provide a wide base. The elongate member 1640 can extend distally from the flange 1620. The first coupling element 1630 can be coupled to a proximal end portion of the elongate member 1640 (e.g., adjacent to the flange 1620) and can have a substantially annular shape. The piercing portion 1660 can extend distally from the elongate member 1640.

FIG. 17 is an enlarged view of a proximal end portion of the obturator 1600. As shown, the flange 1620 can include a second coupling element 1722, which, in the illustrated embodiment, is coupled to a bottom surface of the flange 1620 and is substantially disc-shaped with a cutout or notch 1724. It is appreciated that the first coupling element 1630 and the second coupling element 1722 (including the notch 1724) can be identical or substantially similar in shape, size, function, and/or distance therebetween as compared to the first coupling element 227 and the second coupling element 424 (including the notch 425), respectively. Therefore, when the obturator 1600 is assembled with the second visualization sheath 130, the first coupling element 1630 and the second coupling element 1722 can be disposed in the internal annular groove 833 and in the recess 734, respectively. Thus, the first coupling element 1630 and the second coupling element 1722 can fix both the relative position and orientation of the obturator 1600 and the second visualization sheath 130.

FIG. 18 is an enlarged view of a distal end portion of the obturator 1600. As shown, the piercing portion 1660 can have angled faces, sharp edges and/or points, and/or the like. In particular, the piercing portion 1660 can be sharp enough to stab through tissue, but blunt or dull enough to avoid damaging the spinal cord, nerves, and/or other vulnerable parts of the patient. In some embodiments, the geometry and size of the piercing portion 1660 is customized or selected specifically for a particular patient. Also, in the illustrated embodiment, the elongate member 1640 has a smaller cross-sectional dimension (e.g., diameter) than the piercing portion 1660 so as to form a collar portion 1650 therebetween. The piercing portion 1660 can be sized to fit in the channel 531 of the second visualization sheath 130.

Referring to FIGS. 16-18 together, the obturator 1600 can be assembled with the second visualization sheath 130 by inserting the piercing portion 1660 and the elongate member 1640 through the channel 531. The elongate member 1640 can have a length such that when the obturator 1600 and the second visualization sheath 130 are assembled together, the piercing portion 1660 extends beyond the distal tip 562 of the elongate member 560 (FIG. 5). In operation, a user can insert the assembled obturator 1600 and the second visualization sheath 130 into, e.g., the first port 106 (FIG. 1). The sharp geometry of the piercing portion 1660 can facilitate stabbing through patient tissue. The user can grip the handle 1610 to maneuver the obturator 1600 as needed to create an appropriate access path from the first port 106 to the working space 108. In particular, because the elongate member 1640 has a smaller diameter than the piercing portion 1660, and the channel 531 of the second visualization sheath 130 is sized to receive at least the diameter of the piercing portion 1660, the gap between the walls of the channel 531 and the obturator 1600 can allow the user to bend the elongate member 1640 within the channel 531 to, e.g., stab through tissue at varying angles.

Once the second visualization sheath 130 is at an appropriate depth, the user can remove the obturator 1600 from the second visualization sheath 130 while substantially maintaining the position of the second visualization sheath 130. The gap between the walls of the channel 531 and the elongate member 1640 can facilitate removal of the obturator 1600 by allowing the elongate member 1640 to bend. Subsequently, the user can assemble the first visualization sheath 120 with the second visualization sheath 130, and secure the visualization instrument 112, as shown in FIG. 1.

FIG. 19 is a partially schematic view of a surgical system 1900 configured in accordance with embodiments of the present technology. The surgical system 1900 can share several identical or similar components as the surgical system 100 of FIG. 1. For example, the surgical system 1900 can include a visualization assembly 1910 and the instrument assembly 180. The visualization assembly 1910 can include the visualization instrument 112 having the distal tip 114, the first visualization sheath 120, the first surgical instrument 115 (FIG. 1), the light source 116 operably coupled to the first visualization sheath 120 via the first connector 117, and the first irrigation device 118. As discussed above with reference to FIG. 1, the instrument assembly 180 can include the second irrigation device 182 and the cannula 190.

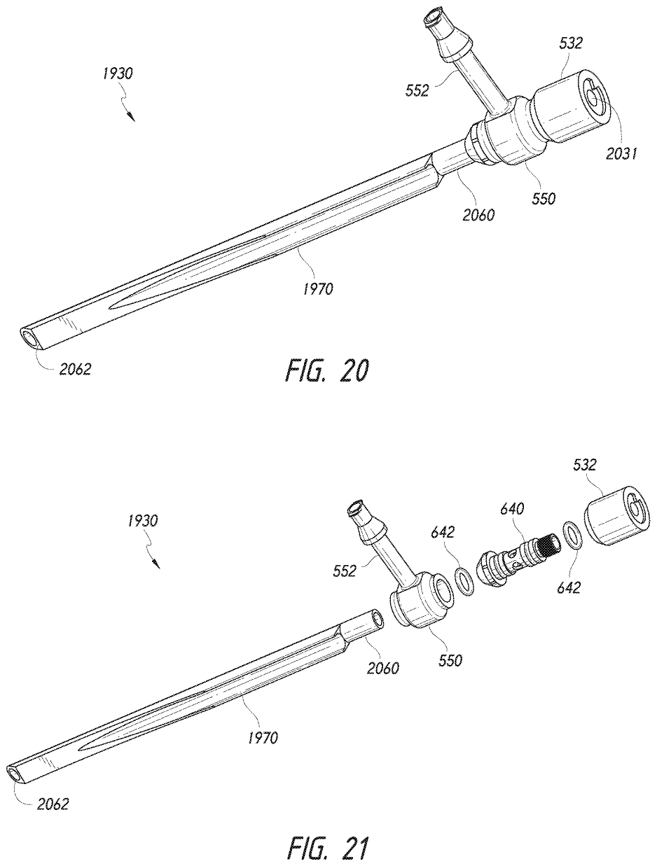

In contrast with the visualization assembly 110 of FIG. 1, however, the visualization assembly 1910 includes a second visualization sheath 1930. The first irrigation device 118 can be operably coupled to the second visualization sheath 1930 via the second connector 119. As discussed in further detail below with reference to FIGS. 20 and 21, the second visualization sheath 1930 can share several identical or similar components as the second visualization sheath 130. In particular, the second visualization sheath 1930 can include an instrument guide 1970. The instrument guide 1970 can be an example of the instrument guide 170 of FIG. 1.

As illustrated in FIG. 19, the cannula 190 can be at least partially inserted through the second port 104 such that the cannula 190 can guide the second surgical instrument 188 through the second port 104 and to the working space 108. Also, the visualization assembly 1910 can be at least partially inserted through the first port 106 such that the distal tip 114 of the visualization instrument 112 can reach the working space 108. In particular, and as described in further detail herein, the first surgical instrument 115 can also be inserted at least partially through the first port 106, and the instrument guide 1970 can guide the first surgical instrument 115 to the working space 108. Therefore, the surgical system 1900 enables simultaneously (i) visualizing the working space 108 (e.g., via the visualization instrument 112), (ii) irrigating the working space 108 through the first port 106 (e.g., via the first irrigation device 118), (iii) irrigating the working space 108 through the second port 104 (e.g., via the second irrigation device 182), (iv) using the first surgical instrument 115 through the first port 106, and/or (v) using the second surgical instrument 188 through the second port 104.

FIGS. 20 and 21 are top isometric and partially exploded isometric views, respectively, of the second visualization sheath 1930. Referring to FIGS. 20 and 21 together, the second visualization sheath 1930 can include the sheath head 532, the sheath body 550 including the laterally protruding tube 552, the irrigation fluid router 640, and the one or more sealing members 642. The second visualization sheath 1930 can also include an elongate member 2060 having a distal tip 2062, and the instrument guide 1970. In some embodiments, the elongate member 2060 and the instrument guide 1970 are integrally formed. In some embodiments, the instrument guide 1970 is permanently or removably coupled to the elongate member 2060. As labeled in FIG. 20, the second visualization sheath 1930, when assembled, can define a channel 2031 extending from the sheath head 532 to the distal tip 2062 of the elongate member 2060, and shaped and sized to receive at least a portion of the first visualization sheath 120, as shown in FIG. 19.

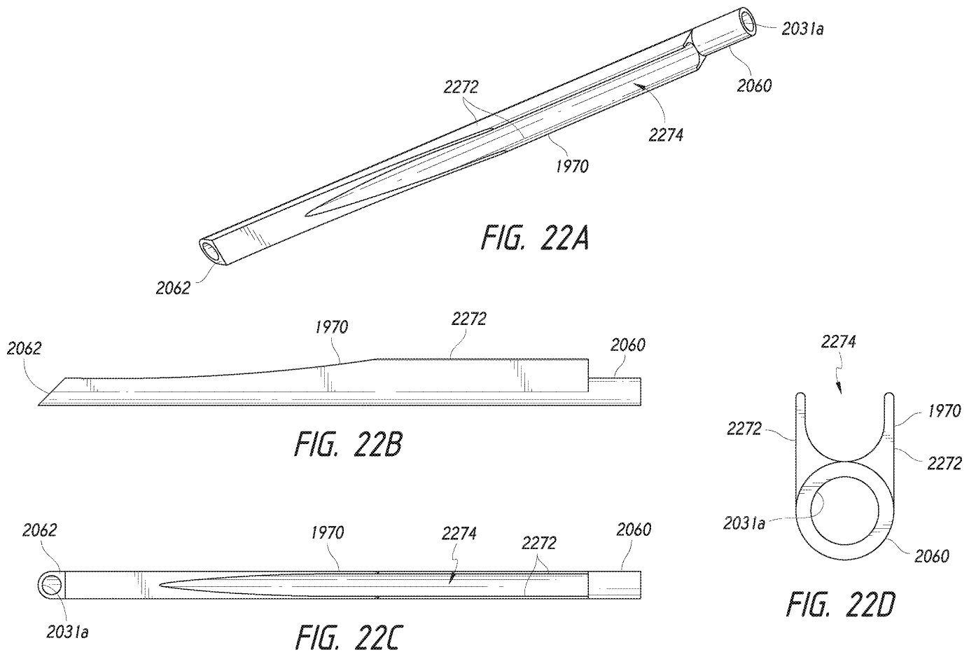

FIGS. 22A-22D are top isometric, side, front, and top views, respectively, of the elongate member 2060 and the instrument guide 1970. Referring to FIGS. 22A-22D together, the elongate member 2060 can be substantially cylindrical in shape and can define a channel portion 2031a extending fully through to the distal tip 2062. The channel portion 2031a can form part of the channel 2031 (FIG. 20) when the second visualization sheath 1930 is fully assembled. In the illustrated embodiment, the instrument guide 1970 extends substantially along the length of the elongate member 2060. The instrument guide 1970 may not extend along the proximal end portion of the elongate member 2060 to allow the elongate member 2060 to be coupled to the irrigation fluid router 640 (e.g., substantially as depicted in FIG. 14). Also, as shown, the instrument guide 1970 can include a pair of opposing, parallel walls 2272 that define a U-shaped instrument-receiving channel 2274 therebetween and extending along an axial length of the elongate member 2060. In particular, the instrument-receiving channel 2274 can be sized to receive the first surgical instrument 115 therethrough. In other words, the instrument guide 1970 can serve as a cannula for the first surgical instrument 115. The pair of walls 2272 can taper toward the distal tip 2062 to facilitate insertion of the second visualization sheath 1930.

In some embodiments, the instrument guide 1970 is modular. For example, the instrument guide 1970 may be detachable from the elongate member 2060 and swapped for another instrument guide having a different shape and/or size. The particular instrument guide can be selected based on, for example, the surgical instrument to be used with the second visualization sheath 1930, the size of the first port 106, and/or the like. The instrument guide 1970 can be configured to hold instruments and can include, without limitation, one or more magnets, biasing members, or the like. For example, the instrument guide 1970 can have one or more magnets configured to magnetically hold an instrument while the instrument is moved distally/proximally along the instrument guide 1970. A user can overcome the magnetic force to move the instrument out of the instrument guide 1970. In some embodiments, sidewalls of the instrument guide 1970 can be biased inwardly to hold on to sides of an instrument. This allows for flexibility when holding is desired during a surgical procedure.

It is appreciated that the illustrated instrument guide 1970 is merely one example of the instrument guide 170, and that instrument guides of other designs that are coupled to and/or formed as a part of, e.g., the elongate member 2060 are within the scope of the present technology. For example, in some embodiments, an instrument guide includes a tubular guiding member defining a closed channel (e.g., as opposed to the open, U-shaped instrument-receiving channel 2274) extending along the length of the elongate member 2060. Furthermore, while not illustrated, in some embodiments, the elongate member 2060 and/or the instrument guide 1970 includes one or more apertures (e.g., the apertures 564 of FIG. 5) at or near the distal tip 2062. Such apertures can provide additional or alternative exit paths for the irrigation fluid.

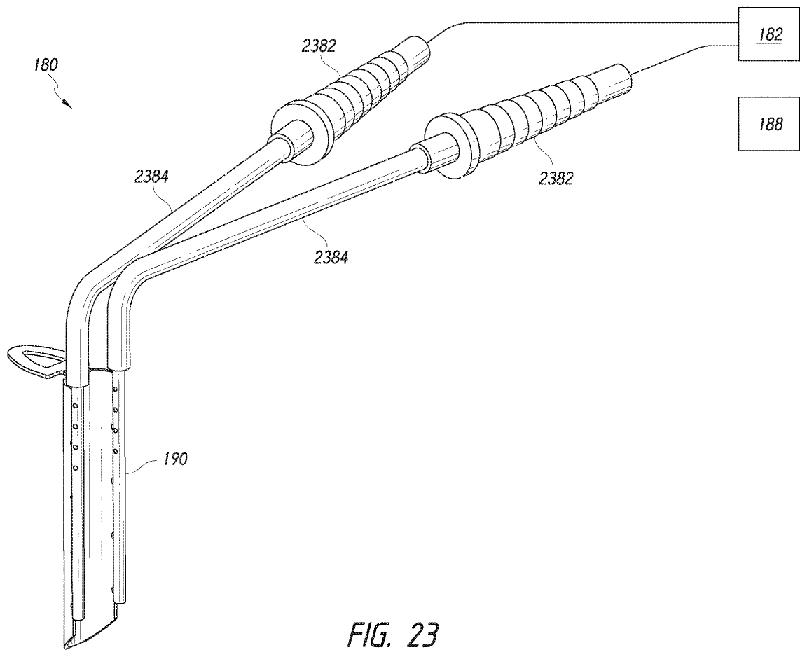

FIG. 23 is an isometric view of the instrument assembly 180. The instrument assembly 180 can include the second irrigation device 182, the second surgical instrument 188, the cannula 190, a pair of adapters 2382, and a pair of tubes 2384. As shown, the second irrigation device 182 can be operably coupled to the cannula 190 via the pair of adapters 2382 and the pair of tubes 2384. The pair of tubes 2384 can each be rigid or flexible, and can be coupled to different parts of the cannula 190. In the illustrated embodiment, the pair of adapters 2382 are each tapered with ridges such that each adapter 2382 can, e.g., fit into a pipe, a port, and/or the like of the second irrigation device 182. Accordingly, the adapters 2382 can couple corresponding ones of the tubes 2384 to the second irrigation device 182. As discussed in further detail herein, in operation, the second irrigation device 182 can pump and/or suck irrigation fluid to and/or from the cannula 190.

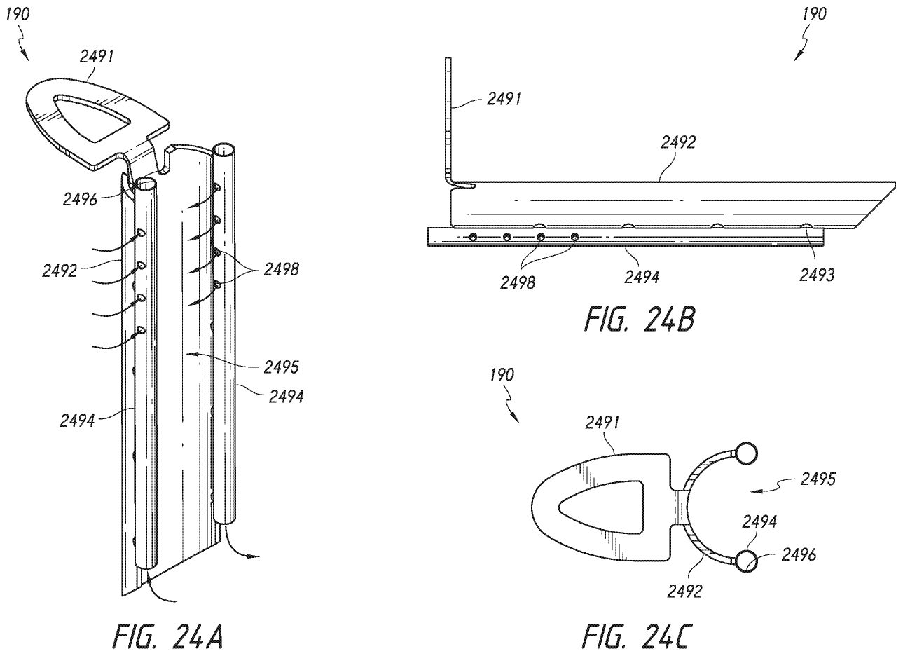

FIGS. 24A-24C are isometric, side, and top views, respectively, of the cannula 190. Referring to FIGS. 24A-24C together, the cannula 190 can include a cannula body 2492, a flange 2491, and a pair of irrigation tubes or pipes 2494. As best seen in FIG. 24C, the cannula body 2492 can be an elongate component with a half-cylindrical cross-section that defines an open channel 2495. In particular, the open channel 2495 can be shaped and sized to receive and guide the second surgical instrument 188 therethrough. As best seen in FIG. 24B, the cannula body 2492 can include one or more notches 2493 along its edges. The notches 2493 can be sized to receive patient tissue and can thereby help secure a position of the cannula 190 within the patient. The flange 2491 can extend from a top or proximal end of the cannula body 2492 along a direction substantially perpendicular to the cannula body 2492. The flange 2491 can serve as a handle for the user and/or abut against the patient's skin to define a maximum insertion depth of the cannula 190.

The irrigation pipes 2494 can be coupled to and extend along the two edges of the cannula body 2492 (e.g., along the notches 2493). In the illustrated embodiment, as best seen in FIG. 24B, the irrigation pipes 2494 can extend beyond the top or proximal end of the cannula body 2492, but not extend fully to the distal end of the cannula body 2492. In other embodiments, however, the irrigation pipes 2494 can have different lengths and/or can be coupled to different points along the cannula body 2492. Each irrigation pipe 2494 can define a channel 2496 and include one or more apertures 2498. Each channel 2496 can extend fully through the corresponding irrigation pipe 2494 along a length thereof. The apertures 2498 can be formed along the length of the corresponding irrigation pipe 2494 and can be in fluid communication with the channel 2496. In the illustrated embodiment, the apertures 2498 are formed along the upper half of the irrigation pipes 2494 and facing either (i) inward toward the open channel 2495 or (ii) outward away from the open channel 2495. It is appreciated that in other embodiments, the apertures 2498 can be formed on the irrigation pipes 2494 in a different arrangement. When suction is applied to the cannula 190, fluid can be drawn into the distal ends of the irrigation pipes 2494. Additionally, irrigation fluid close to the incision can be drawn into the apertures 2498. The captured irrigation fluid can flow proximally along the irrigation pipes 2494 and out of the patient. Suction can be increased or decreased to maintain a desired level of irrigation fluid removal while inhibiting, limiting, or preventing irrigation fluid from flowing out of the patient. Additionally, the patient's tissue can be drawn against the sides of the cannula 190. For example, sufficient suction can be applied to pull the subject's tissue against the sides of the irrigation pipes 2494 to, for example, inhibit, limit, or prevent movement of the cannula 190 relative to the patient. The number, configuration, and spacing of the apertures 2498 can be selected based on the procedure to be performed.

Referring to FIGS. 23-24C together, the second irrigation device 182 can be operable to provide irrigation via the pair of adapters 2382, the pair of tubes 2384, and the pair of irrigation pipes 2494. In particular, the second irrigation device 182 can output irrigation fluid and/or provide suction to retrieve irrigation fluid before, while, or after the cannula 190 guides the second surgical instrument 188. Accordingly, (i) both irrigation pipes 2494 can be used to deliver irrigation fluid to the working space 108, (ii) both irrigation pipes 2494 can be used to withdraw (e.g., via suction) irrigation fluid from the working space 108, or (iii) one irrigation pipe 2494 can be used to deliver irrigation fluid to the working space 108 while the other irrigation pipe 2494 is used to withdraw irrigation fluid from the working space 108.

In some embodiments, the instrument assembly 180 can be used to prepare the working space 108 prior to inserting the visualization assembly 110, 1910. For example, the cannula 190 can first be inserted through the second port 104, then the second irrigation device 182 can be operated to deliver irrigation fluid and thereby inflate a cavity in the patient with the irrigation fluid. Subsequently, the second visualization sheath 130 and the obturator 1600 can be inserted through the first port 106, and the obturator 1600 can pierce the inflated cavity. Accessing the working space 108 in this manner is expected to ensure that the inflated cavity remains an enclosed space in which the flow of the irrigation fluid can be controlled (e.g., without leaking elsewhere).

Referring back to FIGS. 1 and 19, the surgical system 100/1900 can simultaneously or selectively (i) visualize the working space 108 (e.g., via the visualization assembly 110/1910), (ii) provide irrigation of the working space 108 through the first port 106 (e.g., via the visualization assembly 110/1910) and/or through the second port 104 (e.g., via the instrument assembly 180), and/or (iii) operate at the working space 108 through the first port 106 (e.g., via the first surgical instrument 115) and/or through the second port 104 (e.g., via the second surgical instrument 188). Therefore, surgical systems configured in accordance with embodiments of the present technology provide a significantly higher degree of flexibility in performing multi-portal surgical procedures.

It is appreciated that the various features disclosed herein can be used together or independently. For example, the cannula 190 can be used in one port while an endoscope (without the first or second visualization sheath 120, 130) is used in another port. As another example, the visualization assembly 110, 1910 can be used without the cannula 190 and/or the second irrigation device 182.

### III. Select Embodiments of a Method for Operating a Surgical System

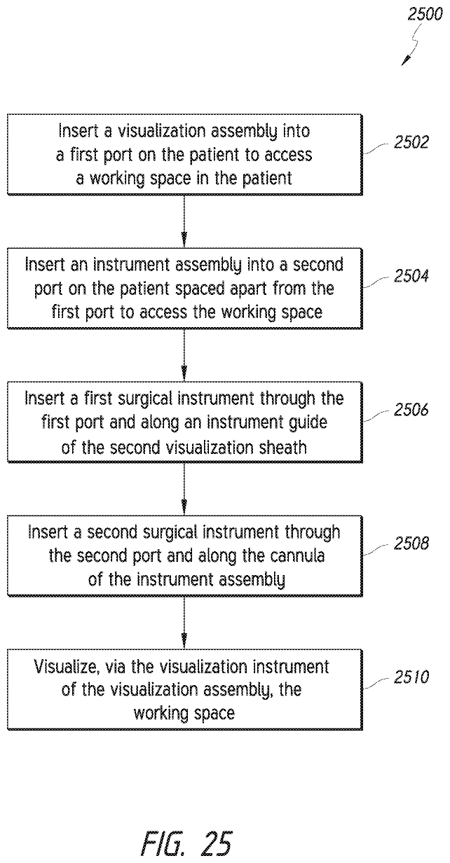

FIG. 25 is a flowchart illustrating a method 2500 for performing a multi-portal spinal procedure on a patient in accordance with some embodiments of the present technology. While the steps of the method 2500 are described below in a particular order, one or more of the steps can be performed in a different order or omitted, and the method 2500 can include additional and/or alternative steps. Additionally, although the method 2500 may be described below with reference to the embodiments of the present technology described herein, the method 2500 can be performed with other embodiments of the present technology.

The method 2500 begins at block 2502 by inserting a visualization assembly (e.g., the visualization assembly 110, 1910) into a first port (e.g., the first port 106) on the patient to access a working space (e.g., the working space 108) in the patient. The visualization assembly can include a visualization instrument (e.g., the visualization instrument 112) configured to visualize the working space in the patient, a first visualization sheath (e.g., the first visualization sheath 120) configured to receive the visualization instrument, and a second visualization sheath (e.g., the second visualization sheath 130) configured to receive the visualization instrument and the first visualization sheath. In some embodiments, inserting the visualization assembly includes (i) extending an obturator through a channel of the second visualization sheath, (ii) forming an access path between the first port and the working space using a piercing portion of the obturator, (iii) removing the obturator from the channel of the second visualization sheath while the second visualization sheath remains in the patient, and (iv) inserting the first visualization sheath into the channel of the second visualization sheath.

At block 2504, the method 2500 continues by inserting an instrument assembly (e.g., the instrument assembly 180) into a second port (e.g., the second port 104) on the patient spaced apart from the first port to access the working space. The instrument assembly can include a cannula (e.g., the cannula 190).

At block 2506, the method 2500 continues by inserting a first surgical instrument (e.g., the first surgical instrument 115) through the first port and along an instrument guide (e.g., the instrument guide 170, 1970) of the second visualization sheath.

At block 2508, the method 2500 continues by inserting a second surgical instrument (e.g., the second surgical instrument 188) through the second port and along the cannula of the instrument assembly.

At block 2510, the method 2500 continues by visualizing, via the visualization instrument of the visualization assembly, the working space. In some embodiments, the method 2500 includes visualizing the working space while the first surgical instrument and the second surgical instrument are simultaneously accessing the working space of the patient.

In some embodiments, the method 2500 further includes irrigating the working space via the second visualization sheath of the visualization assembly while the second visualization sheath is inserted through the first port, and irrigating the working space via the cannula of the instrument assembly while the cannula is inserted through the second port. Irrigating the working space via the cannula can include at least one of (i) delivering irrigation fluid to the working space via a pair of irrigation pipes (e.g., the pair of irrigation pipes 2494) of the cannula, (ii) withdrawing, via a suction force, the irrigation fluid from the working space via the pair of irrigation pipes, or (iii) delivering irrigation fluid to the working space via one of the pair of irrigation pipes and simultaneously withdrawing, via the suction force, the irrigation fluid from the working space via the other of the pair of irrigation pipes.

IV. Conclusion

It will be apparent to those having skill in the art that changes may be made to the details of the above-described embodiments without departing from the underlying principles of the present disclosure. In some cases, well-known structures and functions have not been shown or described in detail to avoid unnecessarily obscuring the description of the embodiments of the present technology. Although steps of methods may be presented herein in a particular order, alternative embodiments may perform the steps in a different order. Similarly, certain aspects of the present technology disclosed in the context of particular embodiments can be combined or eliminated in other embodiments. Furthermore, while advantages associated with certain embodiments of the present technology may have been disclosed in the context of those embodiments, other embodiments can also exhibit such advantages, and not all embodiments need necessarily exhibit such advantages or other advantages disclosed herein to fall within the scope of the technology. Accordingly, the disclosure and associated technology can encompass other embodiments not expressly shown or described herein, and the invention is not limited except as by the appended claims.

Features from various systems, methods, features, and instruments can be combined with systems, methods, features, and instruments disclosed in U.S. application Ser. No. 15/793,950; U.S. application Ser. No. 17/902,685; U.S. application Ser. No. 18/335,737; U.S. application Ser. No. 18/464,949; U.S. application Ser. No. 18/470,140; U.S. application Ser. No. 18/764,784; U.S. application Ser. No. 18/988,467; U.S. application Ser. No. 18/987,830; U.S. Pat. Nos. 8,632,594; 9,308,099; 10,105,238; 10,201,431; 10,898,340; 11,464,648; 11,678,906; 11,950,770; PCT App. No. PCT/US2020/049982; PCT App. No. PCT/US2022/021193; PCT App. No PCT/US2025/014790, which are all hereby incorporated by reference and made a part of this application. To the extent any material incorporated herein by reference conflicts with the present disclosure, the present disclosure controls.

Where the context permits, singular or plural terms may also include the plural or singular term, respectively. For example, throughout this disclosure, the singular terms “a,” “an,” and “the” include plural referents unless the context clearly indicates otherwise. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Furthermore, as used herein, the phrase “and/or” as in “A and/or B” refers to A alone, B alone, and both A and B. Additionally, the terms “comprising,” “including,” “having,” and “with” are used throughout to mean including at least the recited feature(s) such that any greater number of the same features and/or additional types of other features are not precluded. Moreover, as used herein, the phrases “based on,” “depends on,” “as a result of,” and “in response to” shall not be construed as a reference to a closed set of conditions. For example, a step that is described as “based on condition A” may be based on both condition A and condition B without departing from the scope of the present disclosure. In other words, as used herein, the phrase “based on” shall be construed in the same manner as the phrase “based at least in part on” or the phrase “based at least partially on.”

Reference herein to “one embodiment,” “an embodiment,” “some embodiments,” or similar formulations means that a particular feature, structure, operation, or characteristic described in connection with the embodiment can be included in at least one embodiment of the present technology. Thus, the appearances of such phrases or formulations herein are not necessarily all referring to the same embodiment. Furthermore, various particular features, structures, operations, or characteristics may be combined in any suitable manner in one or more embodiments.

Unless otherwise indicated, all numbers expressing numerical values used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present technology. At the very least, and not as an attempt to limit the application of the doctrine of equivalents to the scope of the claims, each numerical parameter should at least be construed in light of the number of reported significant digits and by applying ordinary rounding techniques. The terms “about,” “approximately,” and “substantially” as used herein shall be interpreted to mean within ±10% of the stated value. Additionally, all ranges disclosed herein are to be understood to encompass the endpoints, and any and all subranges subsumed therein. For example, a range of “1 to 10” includes any and all subranges between (and including) the minimum value of 1 and the maximum value of 10 (e.g., any and all subranges having a minimum value of equal to or greater than 1 and a maximum value of equal to or less than 10, such as 5.5 to 10).

The disclosure set forth above is not to be interpreted as reflecting an intention that any claim or example requires more features than those expressly recited in that claim or example. Rather, as the preceding examples and the following claims reflect, inventive aspects lie in a combination of fewer than all features of any single foregoing disclosed embodiment. Thus, the preceding examples and the following claims are hereby expressly incorporated into the Detailed Description, with each claim standing on its own as a separate embodiment. This disclosure includes all permutations of the independent claims with their dependent claims.

What is claimed is:

1. A surgical system for performing multi-portal spinal procedures on a patient, the surgical system comprising:
an endoscopic visualization assembly at least partially insertable through a first port in the patient, the endoscopic visualization assembly including:
a first visualization sheath configured to receive a visualization instrument and to couple to a light source, and
a second visualization sheath configured to be fluidically coupled to an irrigation system such that irrigation fluid from the irrigation system flows along the endoscopic visualization assembly toward a working space, wherein the second visualization sheath is configured to receive the first visualization sheath and includes an instrument guide configured to receive and guide a first surgical instrument positioned outside of the second visualization sheath toward the working space.

2. The surgical system of claim 1, wherein the second visualization sheath includes a tubular member, and wherein the instrument guide has an instrument-receiving channel extending along an axial length of the tubular member.

3. The surgical system of claim 1, wherein the instrument guide includes at least one of (i) a pair of walls defining a U-shaped instrument-receiving channel sized to receive and guide the first surgical instrument or (ii) a tubular guide member defining a closed instrument-receiving channel sized to receive and guide the first surgical instrument.

4. The surgical system of claim 1, wherein the second visualization sheath includes:
a coupler head configured to detachably couple to the first visualization sheath;
a connector body coupled to the coupler head and having a laterally protruding tube configured to receive irrigation fluid;
an elongate tubular member extending distally from the connector body; and
an irrigation fluid router disposed at least partially inside the connector body and coupled between the coupler head and the elongate tubular member.

5. The surgical system of claim 1, wherein:
the first visualization sheath includes:
an elongate member,
a first coupling element coupled to the elongate member and having an annular shape,
a sheath head coupled to the elongate member, and
a second coupling element coupled to a bottom surface of the sheath head, wherein the second coupling element is disc-shaped and includes a notch;
the second visualization sheath includes a sheath head having an internal annular groove, a recess, and a protrusion extending partially into the recess; and
when the first visualization sheath is coupled to the second visualization sheath,
the first coupling element of the first visualization sheath is configured to be positioned in the internal annular groove of the second visualization sheath,
the second coupling element of the first visualization sheath is configured to be positioned in the recess of the second visualization sheath, and
the protrusion of the second visualization sheath is configured to be positioned in the notch of the second coupling element.

6. The surgical system of claim 1, wherein the endoscopic visualization assembly further includes an obturator configured to be coupled to the second visualization sheath and having:
an elongate member sized to fit inside and extend through a channel of the second visualization sheath; and
a piercing portion coupled to a distal end of the elongate member, wherein the piercing portion is sized to fit inside and extend through the channel of the second visualization sheath, and wherein the piercing portion is shaped to stab through tissue.

7. The surgical system of claim 1, wherein the endoscopic visualization assembly further includes a unitary visualization support unit including:
a light source coupleable to the first visualization sheath; and
an irrigation device coupleable to the second visualization sheath.

8. The surgical system of claim 1, further comprising an irrigation cannula assembly at least partially insertable through a second port in the patient spaced apart from the first port, the irrigation cannula assembly including:
an instrument cannula having (i) an open channel configured to receive a second surgical instrument and (ii) one or more irrigation apertures through which irrigation fluid flows into and/or away from the working space;
a pair of irrigation pipes each defining an open channel and having one or more apertures; and
an irrigation device coupleable to each of the irrigation pipes.

9. A method for performing a multi-portal spinal procedure on a patient, the method comprising:
inserting an endoscopic visualization assembly into a first port on the patient to access a working space in the patient, wherein the endoscopic visualization assembly includes:
a visualization instrument configured to endoscopically visualize the working space in the patient,
a first visualization sheath configured to receive the visualization instrument, and
a second visualization sheath configured to receive the visualization instrument and the first visualization sheath;
inserting an irrigation cannula assembly into a second port on the patient spaced apart from the first port to access the working space, wherein the irrigation cannula assembly includes a cannula;
inserting a first surgical instrument through the first port and along an instrument guide of the second visualization sheath;
inserting a second surgical instrument through the second port and along the cannula of the irrigation cannula assembly; and
visualizing, via the visualization instrument of the endoscopic visualization assembly, the working space.

10. The method of claim 9, wherein visualizing comprises visualizing the working space while the first surgical instrument and the second surgical instrument are simultaneously accessing the working space of the patient.

11. The method of claim 9, further comprising:
irrigating the working space via the second visualization sheath of the endoscopic visualization assembly while the second visualization sheath is inserted through the first port; and
irrigating the working space via the cannula of the irrigation cannula assembly while the cannula is inserted through the second port.

12. The method of claim 9, wherein inserting the endoscopic visualization assembly comprises:
extending an obturator through a channel of the second visualization sheath;
forming an access path between the first port and the working space using a piercing portion of the obturator;
removing the obturator from the channel of the second visualization sheath while the second visualization sheath remains in the patient; and
inserting the first visualization sheath into the channel of the second visualization sheath.

13. An endoscopic visualization assembly for use during a spinal procedure on a patient, the endoscopic visualization assembly comprising:
a first visualization sheath having a first channel;
a second visualization sheath having a second channel and an instrument guide, wherein the second channel is sized to receive at least a portion of the first visualization sheath therethrough, and wherein the instrument guide is configured to guide a surgical instrument toward a working space in the patient; and
a visualization instrument sized to at least partially extend through the first channel of the first visualization sheath and configured to visualize the working space.

14. The endoscopic visualization assembly of claim 13, further comprising an irrigation device configured to deliver irrigation fluid to the second channel and around the portion of the first visualization sheath positioned in the second channel.

15. The endoscopic visualization assembly of claim 13, wherein the second visualization sealingly engages the first visualization sheath to define a one-way flow path along the second channel such that irrigation fluid, which is delivered into the second visualization sheath by an irrigation device, flows toward the working space.

16. The endoscopic visualization assembly of claim 13, wherein the first visualization sheath and the second visualization sheath automatically form liquid-tight seals when coupled together.

17. The endoscopic visualization assembly of claim 13, wherein the second visualization sheath includes: a coupler head configured to detachably couple to the first visualization sheath; a connector body coupled to the coupler head and having a laterally protruding tube configured to receive irrigation fluid; an elongate tubular member extending distally from the connector body; and an irrigation fluid router disposed at least partially inside the connector body and coupled between the coupler head and the elongate tubular member.

18. The endoscopic visualization assembly of claim 13, wherein: the first visualization sheath includes an elongate member, a first coupling element coupled to the elongate member and having an annular shape, a sheath head coupled to the elongate member, and a second coupling element coupled to a bottom surface of the sheath head, wherein the second coupling element is disc-shaped and includes a notch; the second visualization sheath includes a sheath head having an internal annular groove, a recess, and a protrusion extending partially into the recess; and when the first visualization sheath is coupled to the second visualization sheath, the first coupling element of the first visualization sheath is positioned in the internal annular groove of the second visualization sheath, the second coupling element of the first visualization sheath is positioned in the recess of the second visualization sheath, and the protrusion of the second visualization sheath is positioned in the notch of the second coupling element.

19. The endoscopic visualization assembly of claim 13, further comprising an obturator configured to be coupled to the second visualization sheath and having an elongate member sized to fit inside and extend through the second channel and a piercing portion coupled to a distal end of the elongate member, wherein the piercing portion is sized to fit inside and extend through the second channel and is shaped to stab through tissue.

20. The endoscopic visualization assembly of claim 13, further comprising a unitary visualization support unit including a light source coupleable to the first visualization sheath and an irrigation device coupleable to the second visualization sheath.
